(19) United States
(12) Patent Application Publication
(10) Pub. No.: US 2005/0165276 A1
(43) Pub. Date: Jul. 28, 2005

(54) Methods and apparatus for accessing and treating regions of the body

(76) Inventors: Amir Belson, Cupertino, CA (US); Robert M. Ohline, Redwood City, CA (US)

Correspondence Address: James Shay;( Wilson Sonsini Goodrich Rosati ), 650 Page Mill Road, Palo Alto, CA, 94304, US

(21) Appl. No.: 10/767,109

(22) Filed: Jan. 28, 2004

(51) Int. Cl. A61B 1/00

(52) U.S. Cl. 600/146

(57) ABSTRACT

Methods and apparatus for accessing and treating regions of the body are disclosed herein. Using an endoscopic device having an automatically controllable proximal portion and a selectively steerable distal portion, the device generally may be advanced into the body through an opening. The distal portion is selectively steered to assume a selected curve along a desired path within the body which avoids contact with tissue while the proximal portion is automatically controlled to assume the selected curve of the distal portion. The endoscopic device can then be used for accessing various regions of the body which are typically difficult to access and treat through conventional surgical techniques because the device is unconstrained by “straight-line” requirements. Various applications can include accessing regions of the brain, thoracic cavity, including regions within the heart, peritoneal cavity, etc., which are difficult to reach using conventional surgical procedures.

### FIELD OF THE INVENTION

The present invention relates generally to endoscopes and endoscopic medical procedures. More particularly, it relates to methods and apparatus for accessing and treating regions within the body which are difficult to reach through conventional surgical devices and procedures.

### BACKGROUND OF THE INVENTION

Many surgical procedures typically require large incisions be made to provide access to regions within the body. For instance, operating on or near the posterior regions of the heart is ordinarily performed using open-chest techniques. Such a procedure generally requires a gross thoracotomy or sternotomy, which are both highly invasive and attendant with a great deal of risks, such as ischemic damage to the heart, formation of emboli, etc. A thoracotomy typically involves creating an incision in the intercostal space between adjacent ribs while a sternotomy involves the “chest spreader” approach, which is generally the most invasive. Moreover, such an invasive procedure produces significant morbidity, increased mortality rates, and significantly increases recovery time for the patient.

Minimally invasive surgery is an alternative surgical procedure in which small incisions are made in the patient's body to provide access for various surgical devices for viewing and operating inside the patient. Laparoscopes are typically used for accessing and performing operations within the body through these small incisions using specially designed surgical instruments. These instruments generally have handles which are manipulatable from outside of the patient's body by the surgeon to control the operation of the instrument typically through an elongated tubular section which fits through a tube, introducer, or trocar device entering the patient's body.

However, even conventional laparoscopic procedures are limited in applicability in part because of a “straight-line” requirement in utilizing laparoscopic tools. This requirement makes accessing certain areas within the body extremely difficult, if not impracticable. Moreover, the lack of flexibility of these tools have made access to certain regions of the body difficult, forcing many surgeons to resort to open surgery rather than utilizing conventional minimally invasive procedures.

Flexible endoscopic devices are also available for use in minimally invasive surgical procedures in providing access to regions within the body. Flexible endoscopes are typically used for a variety of different diagnostic and interventional procedures, including colonoscopy, bronchoscopy, thoracoscopy, laparoscopy and video endoscopy. A flexible endoscope may typically include a fiberoptic imaging bundle or a miniature camera located at the instrument's tip, illumination fibers, one or two instrument channels that may also be used for insufflation or irrigation, air and water channels, and vacuum channels. However, considerable manipulation of the endoscope is often necessary to advance the device through the body, making use of conventional devices more difficult and time consuming and adding to the potential for complications.

Steerable flexible endoscopes have been devised to facilitate selection of the correct path though regions of the body. However, as the device is typically inserted farther into the body, it generally becomes more difficult to advance. Moreover, friction and slack in the endoscope typically builds up at each turn, making it more difficult to advance and withdraw the device. Another problem which may arise, for example, in colonoscopic procedures, is the formation of loops in the long and narrow tube of the colonoscope. Such loops may arise when the scope encounters an obstacle, gets stuck in a narrow passage, or takes on a shape that incorporates compound curves. Rather progressing, the scope forms loops within the patient. In an attempt to proceed in insertion of the colonoscope, for example, excess force may be exerted, damaging delicate tissue in the patient's body. The physician may proceed with the attempted insertion of the endoscope without realizing there is a problem.

Through a visual imaging device the user can observe images transmitted from the distal end of the endoscope. From these images and from knowledge of the path the endoscope has followed, the user can ordinarily determine the position of the endoscope. However, it is difficult to determine the endoscope position within a patient's body with any great degree of accuracy.

None of the instruments described above is flexible enough to address the wide range of requirements for surgical procedures performed internally to the patient's body. Furthermore, the instruments described lack the ability to rotate the distal tip about the longitudinal axis of the instrument while fully articulating the tip to any setting relative to the tubular section of the instrument. This lack of flexibility requires surgeons to manually rotate and move the instrument relative to the patient body to perform the procedure.

### BRIEF SUMMARY OF THE INVENTION

Endoscopic devices, as described below, may be particularly useful in treating various regions within the body. Such endoscopes may include a steerable distal portion and an automatically controlled proximal portion which may be controlled by a physician or surgeon to facilitate steering the device while the proximal portion may be automatically controlled by, e.g., a controller or computer. The steerable endoscope may be advanced within the body of a patient, e.g., via any one of the natural orifices into the body such as through the anus. Alternatively, the device may be introduced percutaneously through a small incision into the body. Once the endoscopic device has been introduced into the body, it may be advanced and maneuvered to avoid obstructing anatomical features such as organs, bones, etc., without impinging upon the anatomy of the patient. Examples of such devices are described in detail in the following patents and co-pending applications: U.S. Pat. No. 6,468,203; U.S. Pat. No. 6,610,007; U.S. patent application Ser. No. 10/087,100filed Mar. 1, 2002; U.S. patent application Ser. No. 10/139,289 filed May 2, 2002, U.S. patent application Ser. No. 10/229,577 filed Aug. 27, 2002; U.S. patent application Ser. No. 10/229,814 filed Aug. 27, 2002, and U.S. patent application Ser. No. 10/306,580 filed Nov. 27, 2002, each of which is incorporated herein by reference in its entirety.

Using such a device, one method of treating an obstructed region of tissue within a body, may generally comprise advancing an elongate device into the body through an opening, the elongate device having a proximal portion and a selectively steerable distal portion and the elongate device having a plurality of segments, selectively steering the distal portion to assume a selected curve along a desired path within the body which avoids contact with tissue (or does not require displacement of adjacent tissue along the desired path or avoids applying excess force to the adjacent tissue), and further advancing the elongate device through the body and towards the region of tissue to be treated while controlling the proximal portion of the device to assume the selected curve of the distal portion.

Using any one of the controllable endoscopic devices, various regions of the body which are typically difficult to access and treat through conventional surgical techniques, may be accessed and treated accordingly. In one treatment variation, the endoscopic device may be utilized for neurological surgical applications. Because the endoscopic device is unconstrained by “straight-line” requirements for accessing regions of the brain which are conventionally difficult to reach and/or because the device avoids forming loops when advanced, the endoscope may be accurately advanced and positioned within the cranium by steering the device around the brain with minimal or no trauma to healthy brain tissue. The endoscope may also be advanced through the tissue as necessary to access treatment areas embedded deep within the tissue through pathways which may minimize any damage to healthy adjacent tissue. Furthermore, because the endoscopic device may allow access to sensitive regions over or within the brain, minimally invasive surgery may be performed where conventional surgery would normally require removal of portions of the skull, for instance, in craniotomy procedures or treatment of intracranial hematomas, etc. In addition, access through the nasal passages or other natural cranial orifices may be facilitated.

Another area of treatment in which the endoscopic device may be utilized may include use for coronary procedures, e.g., treatment of the mitral valve, tissue ablation for the treatment of atrial fibrillation, placement, removal, or adjustment of pacing leads, etc. In one example, the endoscopic device may be introduced within the heart via the superior vena cava and advanced through the right atrium. Once the endoscope is within the right atrium, the distal portion may be steered through the atrial septum and into the left atrium where the distal portion of the device may be positioned adjacent to the tissue to be treated, in this example, the mitral valve. To affect treatment, various tools or devices, e.g., scalpels, graspers, etc., may be delivered through one or several working channel within the device to effect the treatment.

In yet another area of treatment in which the endoscopic device may be utilized, various thoracoscopy procedures may be accomplished in a minimally invasive procedure, e.g., percutaneously. As shown, the endoscope may be advanced into the patient via an introducer or port, which may also be configured as a datum for establishing a fixed point of reference for the endoscope during the procedure. The port or datum may be in electrical communication with a computer or processor used for determining and/or maintaining the position of the device within the patient. The endoscope may be advanced into the body of the patient through an incision made, e.g., in the intercostal space between the ribs. The endoscope may then be advanced into the thoracic cavity and maneuvered to regions within the body such as the posterior region of the heart which are normally inaccessible for conventional laparoscopic procedures due to a lack of straight-line access.

The endoscope device may also be utilized for procedures within the peritoneal cavity. Potential applications may include minimally invasive surgery for urologic, bariatric, and liver surgery. Moreover, minimally invasive access may be achieved for treatments in spinal or orthopedic surgery as well. In such a procedure, the endoscope may be introduced into the patient through an incision via a port, which may also function as a datum. The distal portion may be steered to avoid various organs while being advanced to a tissue region to be treated, e.g., the liver. The distal portion of the endoscope may accordingly be steered while the proximal portion may be automatically controlled to follow a path defined by the distal portion which minimizes contact with the surrounding and adjacent tissue and organs. In this or any other procedure, one or more laparoscopes may optionally be used in combination with the endoscope to assist with the surgical procedure.

### BRIEF DESCRIPTION OF THE DRAWINGS

FIG. 1 shows one variation of a steerable endoscope which may be utilized for accessing various regions within the body without impinging upon the anatomy of the patient.

FIG. 2A shows a wire frame model of a section of the elongate body of the endoscope in a neutral or straight position.

FIG. 2B shows an illustration of the endoscope body maneuvered through a curve with the selectively steerable distal portion and automatically controlled proximal portion.

FIG. 3 shows a cross-sectional side view of a patient's head with a variation of the endoscope being advanced therethrough.

FIG. 4 shows a cross-sectional anterior view of a heart with the endoscopic device introduced via the superior vena cava and advanced to the right atrium.

FIG. 5 shows an example of a thoracoscopy procedure which may be performed percutaneously with the endoscopic device.

FIGS. 6A to 6D show an example of the endoscopic device advanced to the posterior region of a heart for the treatment of atrial fibrillation.

FIG. 7 shows another example of a treatment for atrial fibrillation using the endoscopic device.

FIG. 8 shows yet another example of a treatment for atrial fibrillation using the endoscopic device.

FIG. 9 shows an example of a procedure within the peritoneal cavity which may be performed with the endoscopic device.

FIGS. 10A to 10C shows side and end views, respectively, of various electrode configurations on the endoscope for tissue ablation treatment.

### DETAILED DESCRIPTION OF THE INVENTION

In treating various regions within the body, a number of different endoscopic devices may be utilized in facilitating access. Endoscopic devices which are particularly useful may include various endoscopes having a steerable distal portion and an automatically controlled proximal portion. Generally, the steerable distal portion may be controlled by a physician or surgeon to facilitate steering the device while the proximal portion may be automatically controlled by, e.g., a controller or computer. The steerable endoscope may be advanced within the body of the patient through a number of different methods. For instance, the endoscope may be introduced via any one of the natural orifices into the body such as through the anus. Alternatively, the device may be introduced percutaneously through a small incision into the body. Once the endoscopic device has been introduced into the body, it may be advanced and maneuvered, as described below, to avoid obstructing anatomical features such as organs, bones, etc., without impinging upon the anatomy of the patient.

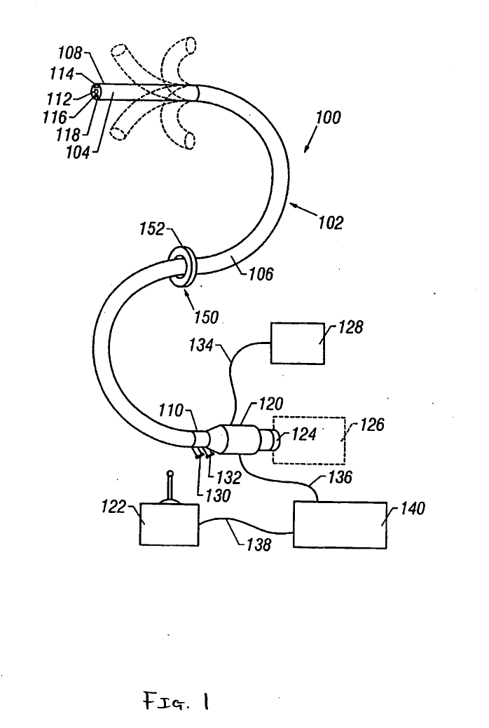

FIG. 1 illustrates one variation of a steerable endoscope 100 which may be utilized for accessing various regions within the body without impinging upon the anatomy of the patient. The endoscope 100 generally has an elongate body 102 with a manually or selectively steerable distal portion 104 and an automatically controlled proximal portion 106. The selectively steerable distal portion 104 may be selectively steered or bent up to a full 180° bend in any direction, as shown by the dashed lines. A fiberoptic imaging bundle 112 and one or more illumination fibers 114 may optionally be extended through the body 102 from the proximal end 110 to the distal end 108. Alternatively, the endoscope 100 may be configured as a video endoscope with a miniaturized video camera, such as a CCD or CMOS camera, positioned at the distal end 108 of the endoscope body 102. The images from the video camera may be transmitted to a video monitor by a transmission cable or by wireless transmission. Optionally, the body 102 of the endoscope 100 may also include at least one or two instrument channels 116, 118 that may be used to provide access through the endoscope for any number of tools. Channels 116, 118 may also be used for various other purposes, e.g., insufflation or irrigation.

The elongate body 102 of the endoscope 100 is highly flexible so that it is able to bend around small diameter curves without buckling or kinking. The elongate body 102 of the endoscope 100 may range in length typically from, e.g., 135 to 185 cm, and 12 to 13 mm in diameter. However, if the endoscope 100 were utilized in regions within the body which are smaller than the space within, e.g., the gastrointestinal tract, the device may be modified in size to be smaller in diameter. The endoscope 100 may also be modified in length to be longer or shorter, depending upon the desired application.

A handle 120 is attachable to the proximal end 110 of the elongate body 102. The handle 120 may include an ocular 124 connected to the fiberoptic imaging bundle 112 for direct viewing and/or for connection to a video camera 126. The handle 120 may also be connected to an illumination source 128 via an illumination cable 134 that may connected to or continuous with the illumination fibers 114. An optional first luer lock fitting 130 and an optional second luer lock fitting 132, which may be in communication with instrument channels 116, 118, respectively, may also be located on or near the handle 120.

The handle 120 may be connected to an electronic motion controller 140 by way of a controller cable 136. A steering control 122 may be connected to the electronic motion controller 140 by way of a second cable 138. The steering control 122 may be configured to allow the physician or surgeon to selectively steer or bend the selectively steerable distal portion 104 of the elongate body 102 in the desired direction. The steering control 122 may be a joystick controller as shown, or other known steering control mechanism. Alternatively, the steering may be effected manually, e.g. by the use of cables, hydraulics, or pneumatics, or any other known mechanical apparatus for controlling the distal portion of the elongate body. The electronic motion controller 140 may be used to control the motion of the automatically controlled proximal portion 106 of the elongate body 102 and may be implemented using a motion control program running on a microcomputer or through an application-specific motion controller. Alternatively, the electronic motion controller 140 may be implemented using a neural network controller.

An axial motion transducer 150 may be provided to measure the axial motion of the elongate body 102 as it is advanced and withdrawn. The axial motion transducer 150 can be made in many configurations, some of which are described below. In this variation, the axial motion transducer 150 is configured as a ring 152, for illustrative purposes only, that surrounds the elongate body 102 of the endoscope 100. The axial motion transducer 150 may be attached to a fixed point of reference, such as the surgical table or the insertion point for the endoscope 100 on the patient's body, as described below. As the body 102 of the endoscope 100 slides through the axial motion transducer 150, it produces a signal indicative of the axial position of the endoscope body 102 with respect to the fixed point of reference and sends a signal to the electronic motion controller 140 by telemetry or by a cable (not shown). The axial motion transducer 150 may use optical, electronic, magnetic, mechanical, etc., methods to determine the axial position of the endoscope body 102. In addition, the motion transducer may be configured to simultaneously measure and communicate rotational motion of the endoscope, so that this additional data may be used in the control of the instrument's motion. A further detailed description for the axial motion transducer 150 and variations thereof may be found in U.S. patent application Ser. No. 10/384,252 filed Mar. 7, 2003, which is incorporated herein by reference in its entirety.

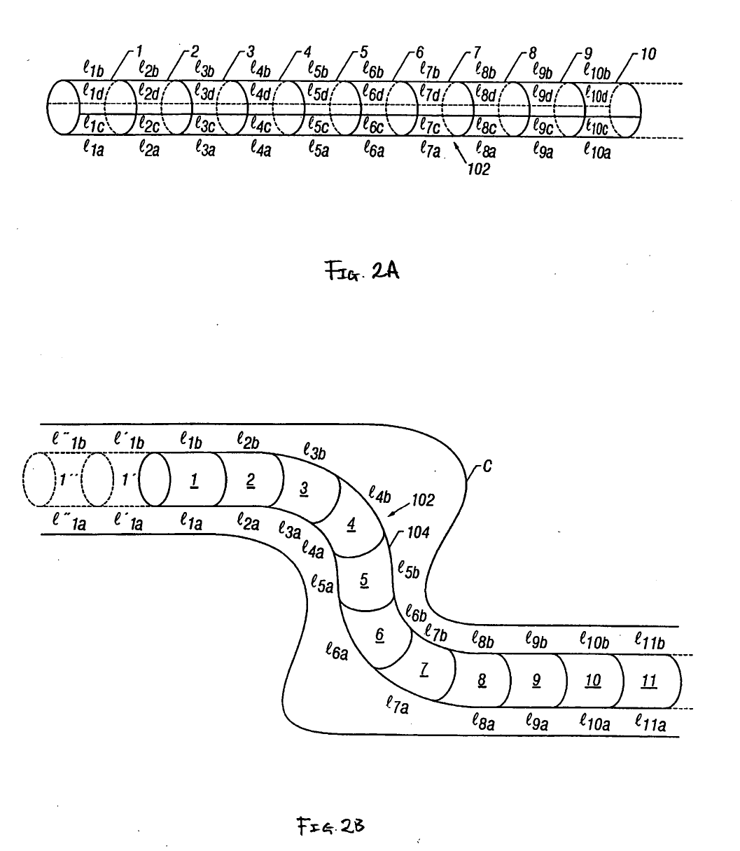

To illustrate the basic motion of the endoscope 100, FIG. 2A shows a wire frame model of a section of the body 102 of the endoscope 100 in a neutral or straight position. Most of the internal structure of the endoscope body 102 has been eliminated in this drawing for the sake of clarity. The endoscope body 102 is divided up into sections 1, 2, 3 . . . 10, etc. The geometry of each section is defined by four length measurements along the a, b, c and d axes. For example, the geometry of section 1 may be defined by the four length measurements l₁ₐ, l_{1b}, l_{1c}, l_{1d}, and the geometry of section 2 may be defined by the four length measurements l₂ₐ, l_{2b}, l_{2c}, l_{2d}, etc. The geometry of each section may be altered using the linear actuators to change the four length measurements along the a, b, c and d axes. For example, to bend the endoscope body 102 in the direction of the a axis, the measurements l₁ₐ, l₂ₐ, l₃ₐ . . . l₁₀ₐ can be shortened and the measurements l_{1b}, l_{2b}, l_{3b} . . . l_{10b} can be lengthened an equal amount. The amount by which these measurements are changed determines the radius of the resultant curve. In the automatically controlled proximal portion 106, however, the a, b, c and d axis measurements of each section may be automatically controlled by the electronic motion controller 140.

In FIG. 2B, the endoscope body 102 has been maneuvered through the curve C with the benefit of the selectively steerable distal portion 104 and now the automatically controlled proximal portion 106 resides in the curve C. Sections 1 and 2 are in a relatively straight part of the curve C, therefore 1₁ₐ=1_{1b} and 1₂ₐ=1_{2b}. However, because sections 3-7 are in the S-shaped curved section, 1₃ₐ<1_{3b}, 1₄ₐ<1_{4b} and 1₅ₐ<1_{5b}, but 1₆ₐ>1_{6b}, 1₇ₐ>1_{7b} and 1₈ₐ>1_{8b}. When the endoscope body 10 advanced distally by one unit, section 1 moves into the position marked 1′, section 2 moves into the position previously occupied by section 1, section 3 moves into the position previously occupied by section 2, etc. The axial motion transducer 150 produces a signal indicative of the axial position of the endoscope body 102 with respect to a fixed point of reference and sends the signal to the electronic motion controller 140. Under control of the electronic motion controller 140, each time the endoscope body 102 advances one unit, each section in the automatically controlled proximal portion 106 is signaled to assume the shape of the section that previously occupied the space that it is now in. Therefore, when the endoscope body 102 is advanced to the position marked 1′, 1₁ₐ=1_{1b}, 1₂ₐ=1_{2b}, 1₃ₐ=1_{3b}, 1₄ₐ<l_{4b}, 1₅ₐ<1_{5b}, 1₆ₐ<1_{6b}, 1₇ₐ>1_{7b}, 1₈ₐ>1_{8b}, and 1₉ₐ>1_{9b}, and, when the endoscope body 102 is advanced to the position marked 1″, 1₁ₐ=1_{1b}, 1₂ₐ=1_{2b}, 1₃ₐ=1_{3b}, 1₄ₐ=1_{4b}, 1₅ₐ<1_{5b}, 1₆ₐ<1_{6b}, 1₇ₐ<1_{7b}, 1₈ₐ>1_{8b}, 1₉ₐ>1_{9b}, and 1₁₀ₐ>1_{10b}. Thus, the S-shaped curve propagates proximally along the length of the automatically controlled proximal portion 106 of the endoscope body 102. The S-shaped curve appears to be fixed in space, as the endoscope body 102 advances distally.

Similarly, when the endoscope body 102 is withdrawn proximally, each time the endoscope body 102 is moved proximally by one unit, each section in the automatically controlled proximal portion 106 is signaled to assume the shape of the section that previously occupied the space that it is now in. The S-shaped curve propagates distally along the length of the automatically controlled proximal portion 106 of the endoscope body 102, and the S-shaped curve appears to be fixed in space, as the endoscope body 102 withdraws proximally.

Whenever the endoscope body 102 is advanced or withdrawn, the axial motion transducer 150 may be used to detect the change in position and the electronic motion controller 140 may be used to propagate the selected curves proximally or distally along the automatically controlled proximal portion 106 of the endoscope body 102 to maintain the curves in a spatially fixed position. Similarly, if the endoscope 102 is rotated, a rotational motion transducer (separate from or integrated within transducer 150) may be used to detect the change in position and the electronic motion controller may be similarly used to adjust the shape of the endoscope body 102 to maintain the curves in a spatially fixed position. This allows the endoscope body 102 to move through tortuous curves without putting unnecessary force on the wall of the curve C.

Examples of other endoscopic devices which may be utilized in the present invention are described in further detail in the following patents and co-pending applications, U.S. Pat. No. 6,468,203; U.S. Pat. No. 6,610,007; U.S. patent application Ser. No. 10/087,100 filed Mar. 1, 2002; U.S. patent application Ser. No. 10/139,289 filed May 2, 2002, U.S. patent application Ser. No. 10/229,577 filed Aug. 27, 2002; U.S. patent application Ser. No. 10/229,814 filed Aug. 27, 2002, and U.S. patent application Ser. No. 10/306,580 filed Nov. 27, 2002, each of which has been incorporated herein by reference above.

Therefore, using any one of the controllable endoscopic devices described above, various regions of the body which are typically difficult to access and treat through conventional surgical techniques, may be accessed and treated accordingly. In one treatment variation, the endoscopic device may be utilized for neurological surgical applications. Because the endoscopic device is unconstrained by “straight-line” requirements for accessing regions of the brain which are conventionally difficult to reach, the endoscope may be advanced and positioned within the cranium by steering the device around the brain with minimal or no trauma to healthy brain tissue. The endoscope may also be advanced through the tissue as necessary to access treatment areas embedded deep within the tissue through pathways which may minimize any damage to healthy adjacent tissue. Furthermore, because the endoscopic device may allow access to sensitive regions over or within the brain, minimally invasive surgery may be performed where conventional surgery would normally require removal of portions of the skull, for instance, in craniotomy procedures or treatment of intracranial hematomas, etc.

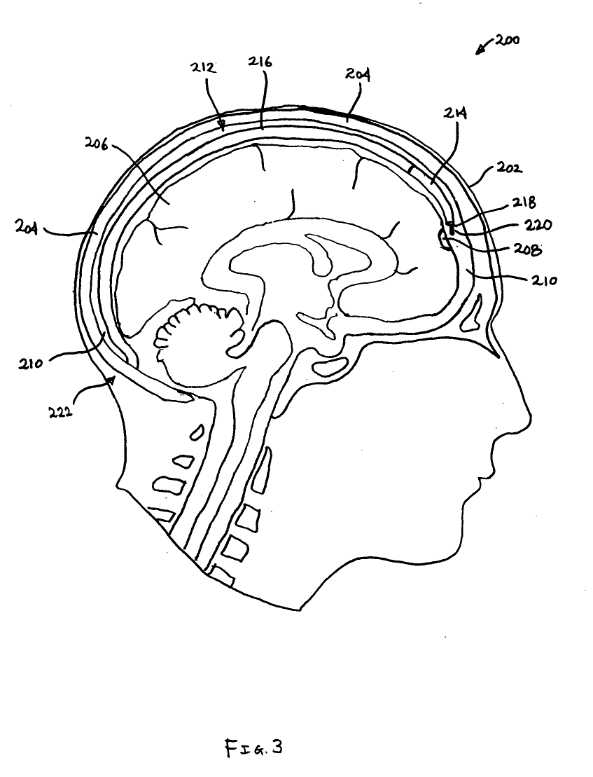

FIG. 3 shows a cross-sectional side view of head 202 of patient 200. The brain 206 may be seen within the cranial cavity 210 of cranium 204. In treating regions of the brain 206 which may be difficult to normally access, the endoscopic device 212 may be introduced into the cranial cavity 210 from an easily accessible insertion site 222, e.g., a perforation within the skull. The endoscope 212 may be then advanced through the insertion site 222 by controlling the steerable distal portion 214 to avoid brain tissue. As the endoscope 212 is further advanced into the cranial cavity 210, the automatically controlled proximal portion 216 may attain the shape defined by the steerable distal portion 214 to avoid contact with brain tissue 206.

The endoscope 212 may be further advanced through the cranial cavity 210 and within the cerebrospinal fluid so that the device is advanced above or within the layers of the meninges, e.g., within the subarachnoid space. In either case, the endoscope 212 may be steered along a path which avoids or minimizes contact or pressure against the brain tissue 206. As the controlled proximal portion 216 is advanced distally and attains the shape defined by the distal portion 214, the proximal portion 216 likewise may be controlled to automatically avoid or minimize contact or pressure against the brain tissue 206. Once the distal portion 216 is advanced to the desired treatment region 208, various tools 220 may be introduced through the instrument channel 218 to enable treatment of the region 208. Any number of treatments or procedures may accordingly be effected, e.g., tumor biopsy and/or removal, shunt placement, lead placement, device placement, drainage of excess cerebrospinal fluid or blood, etc.

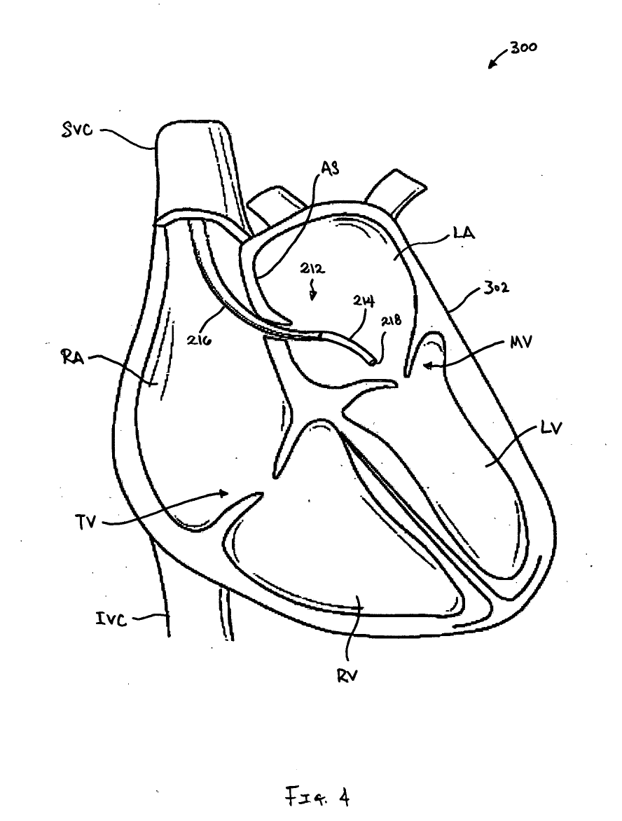

Another area of treatment in which the endoscopic device may be utilized may include use for coronary procedures, e.g., treatment of the mitral valve, tissue ablation for the treatment of atrial fibrillation, the placement, repositioning or removal of device leads, etc. As shown in FIG. 4, a cross-sectional anterior view of heart 302 may be seen in coronary procedure 300 for treatment of the mitral valve MV located between the left atrium LA and the left ventricle LV. The endoscopic device 212 is shown in this treatment variation as being introduced within the heart 302 via the superior vena cava SVC and advanced through the right atrium RA. Also shown is the right ventricle RV below the tricuspid valve TV and inferior vena cava IVC. The endoscope 212 may be sized accordingly to be delivered intravascularly. Once the endoscopic device 212 is within the right atrium RA, the distal portion 214 may be steered towards the atrial septum AS which separates the left atrium LA and right atrium RA. Once at the atrial septum AS, a cutting tool deliverable through the device 212 may be used to perforate the atrial septum AS to allow passage of the endoscopic device 212 into the left atrium LA. The distal portion 214 may then be steered and positioned adjacent the mitral valve MV while the proximal portion 216 is automatically controlled to minimize any pressure which may be exerted by the device 212 against the tissue of the heart 302. Once the endoscopic device is adjacent to the tissue to be treated, in this example the mitral valve MV, various tools or devices may be delivered through the channel 218 to effect the treatment. Once the procedure has been completed, the endoscope 212 may simply be withdrawn proximally in the same manner while minimizing any contact pressure against the tissue.

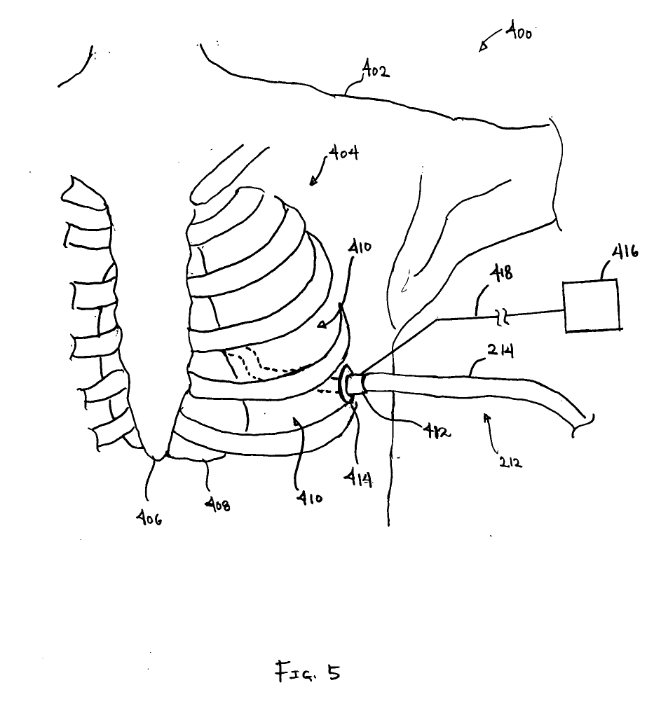

In yet another area of treatment in which the endoscopic device may be utilized, various thoracoscopy procedures may be accomplished in a minimally invasive procedure. FIG. 5 shows an example of a thoracoscopy procedure 400 which may be performed percutaneously. As shown, the endoscope 212 may be advanced into the patient 402 via an introducer or port 412, which may also be configured as a datum for establishing a fixed point of reference for the endoscope 212 during the procedure. The port or datum 412 may be in electrical communication via electrical lines 418 with a computer or processor 416 which may be used for determining and/or maintaining the position of the device 212 within the patient 402. The endoscope 212 may be advanced into the body of the patient 402 through an incision 414 made, e.g., in the intercostal space between the ribs 404. The endoscope 212 may then be advanced into the thoracic cavity and maneuvered to regions within the body such as the posterior region of the heart 408 which are normally inaccessible for conventional laparoscopic procedures due to a lack of straight-line access.

In this example, the endoscopic device 212 is shown having been inserted through port or datum 412 and advanced posteriorly of heart 408 behind sternum 406. The lungs are not shown for the sake of clarity; however, the endoscope 212 may be steered and advanced around the lungs in a manner described above so as to avoid contact or to minimize contact with the lung tissue or any other organs or structures which may be obstructing a straight-line path.

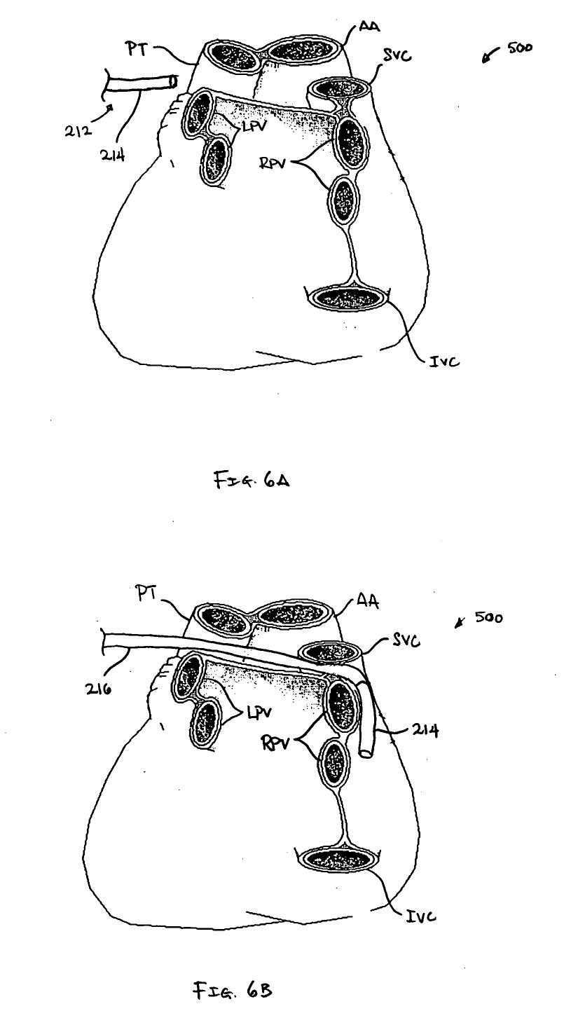

The endoscopic device 212 is capable of reaching regions within the body, without damaging surrounding tissue, which is normally inaccessible via conventional laparoscopic procedures. Yet another procedure 500 is shown in FIGS. 6A to 6D, which illustrate how the endoscopic device may be utilized for the treatment of atrial fibrillation. The figures show a posterior view of the heart with the aorta AA and pulmonary trunk PT as anatomical landmarks. Atrial fibrillation is typically sustained by the presence of multiple electrical reentrant wavelets propagating simultaneously in the atria of the heart. Surgical and catheter-based techniques typically place segmented or continuous lesions near and around the pulmonary veins as one way to re-synchronize the atria.

The endoscopic device 212 may be utilized by advancing the device 212 into the thoracic cavity, as described above or through various other channels, and steered towards the posterior region of the heart. In the example shown in FIGS. 6A to 6D, the steerable distal portion 214 may be advanced as shown in FIG. 6A such that the endoscope 212 approaches above the left pulmonary veins LPV. As shown in FIG. 6B, the distal portion 214 may be steered around the right pulmonary veins RPV while the endoscope 212 is advanced distally. The automatically controllable proximal portion 216 may thus assume the shape defined by the distal portion 214 in traversing around the pulmonary vessels. As shown in FIG. 6C, the distal portion 214 is steered around the left pulmonary vessels LPV while the proximal portion has assumed the curved path traversed by the device around the right pulmonary vessels RPV. Finally in FIG. 6D, the device 212 may be fully advanced entirely around the pulmonary vessels such that the distal portion 214 and proximal portion 216 are in intimate contact against the heart tissue while maintaining its configuration. The tissue which is in contact against the device 212 may then be ablated by one or several electrodes located along the length of the distal and/or proximal portions 214, 216, as described in further detail below. Alternately, an ablation device such as a catheter or other energy source, may be delivered through one or more working channels in or on the endoscope, and left in place as desired. This ablation device may then be used to deliver ablative energy in various forms, e.g., RF, microwave, cryogenic cooling, etc. The device may be held fixedly in the desired location by various methods, e.g., vacuum, magnetically, temporary adhesives, sutures, or any other methods of attaching or approximating the device and tissue.

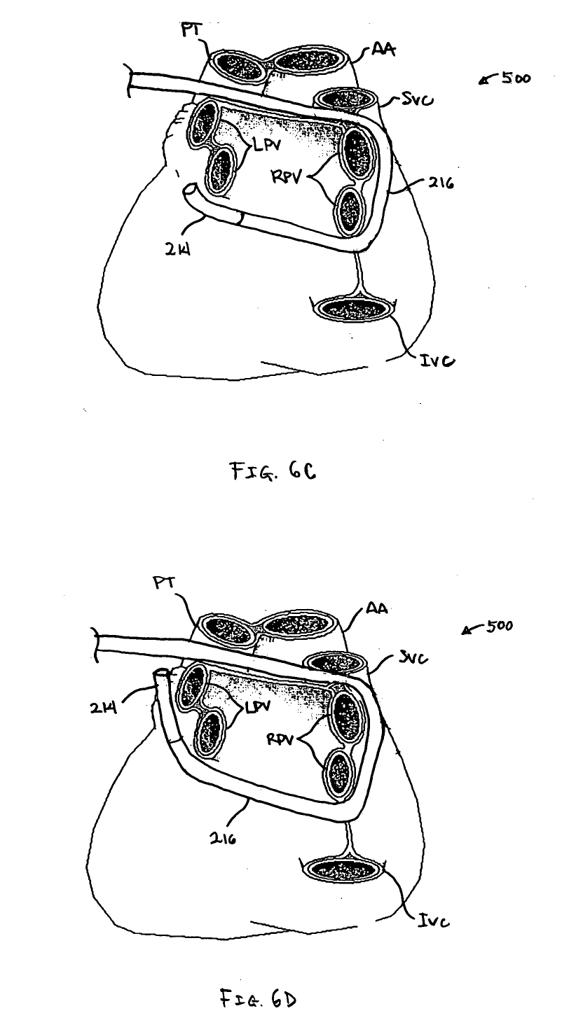

FIG. 7 shows another variation 600 of treating atrial fibrillation where the device may be steered and configured to loop in a continuous manner about the pulmonary vessels in a first encirclement 602 over the left pulmonary vessels LPV and a second encirclement 604 over the right pulmonary vessels RPV. The encircled portions 602, 604 of the endoscope 212 may be activated to ablate the heart tissue only around the pulmonary vessels LPV, RPV or alternatively, it may be activated to ablate the heart tissue along the entire length of both distal portion 214 and proximal portion 216. Moreover, a variety of ablation devices may be delivered to the desired areas, as described above.

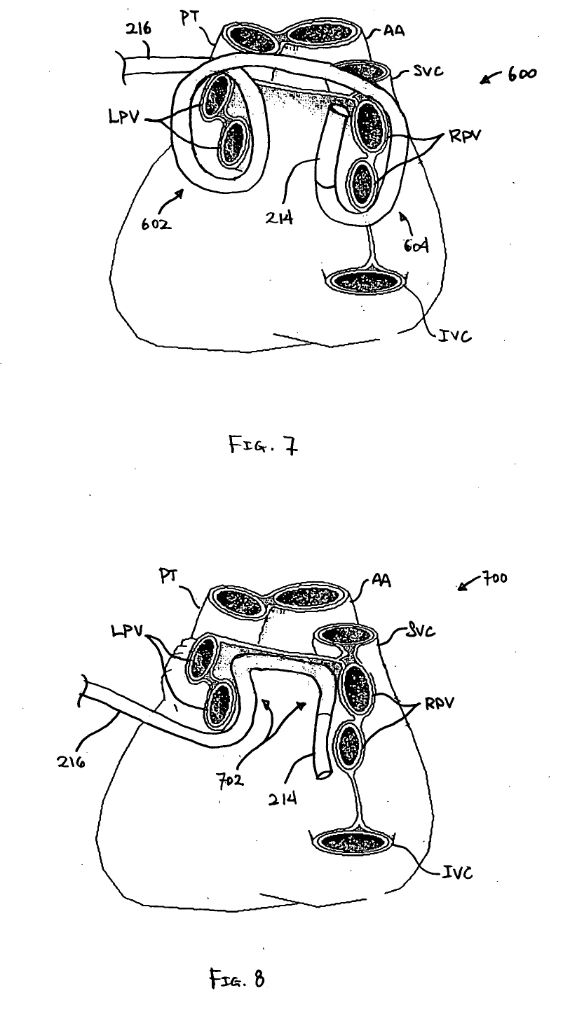

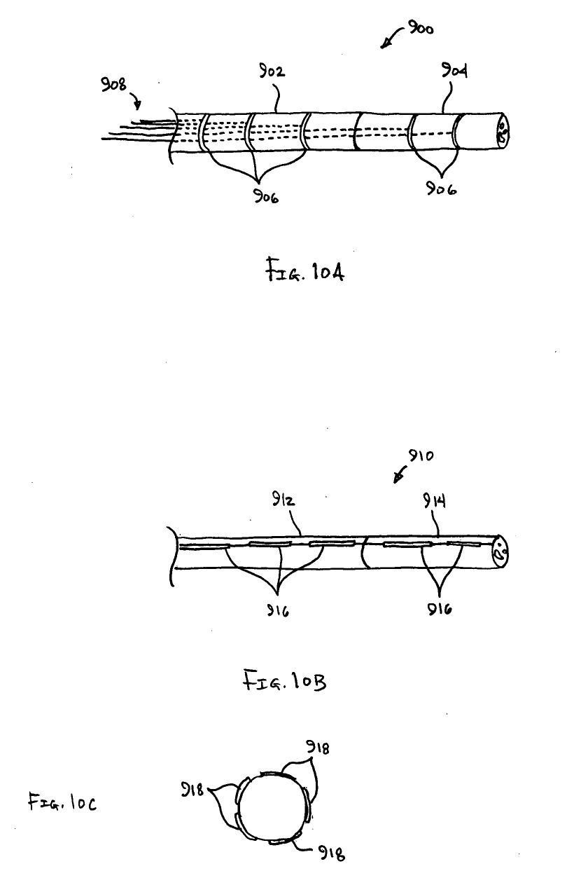

FIG. 8 shows yet another variation 700 in which the endoscope 212 may be advanced and steered to contact the portions of tissue posteriorly adjacent to the pulmonary vessels LPV, RPV such that an encircled region is formed 702. The endoscope 900 may be configured with a number of electrodes over its outer surface to facilitate the tissue ablation along the length, or selected regions of length, of the endoscope, as shown in FIG. 10A. The figure shows the steerable distal portion 904 and part of the automatically controllable proximal portion 902 as one example of electrode placement over the endoscope 900. As seen, one or any number of electrodes 906 may be circumferentially positioned, e.g., ring-shaped, along the length of endoscope 900 at intervals. The electrodes 906 are shown positioned at uniform intervals in this variation; however, they may be configured in any random, arbitrary, or specified locations over the outer surface of the endoscope 900. Each of the electrodes 906 may be electrically connected via corresponding wires 908 to a power supply and/or controller. Thus, all the electrodes 906 may be configured to operate simultaneously or to operate only selected electrodes 906 which may be in contact with tissue. In yet another variation, various ablation devices may be delivered to the desired areas, again as described above.

FIG. 10B shows another variation in endoscope 910 in which electrodes 916 may be configured to extend longitudinally over the proximal portion 912 and/or distal portion 914. The electrodes may be configured to extend in a continuous strip along the endoscope length or the electrodes 916 may be alternatively configured to extend in a segmented manner longitudinally over the endoscope 910, as shown. Having segmented electrodes 916 may allow for selected electrodes to be activated during tissue ablation. Although FIG. 10B shows a single line of electrodes 916 for illustration purposes, multiple lines of electrodes may be positioned over the outer surface of the device, as shown in the example of FIG. 10C, which illustrates multiple lines of electrodes 918 spaced uniformly around the circumference of the endoscope surface.

These examples described above are intended to be illustrative and are not intended to be limiting. Any number of other configurations may be accomplished with the endoscopic device due to the ability of the device to steer and configure itself such that excessive contact with surrounding tissue is avoided. Moreover, access to any number of various regions within the thoracic cavity with minimal or no damage to surrounding tissue and organs may be accomplished using the controllable endoscopic device above. Other examples for treatment using the endoscope may include, but not limited to, lead placement, implantable device placement, treatment on the lungs such as emphysema treatments, etc.

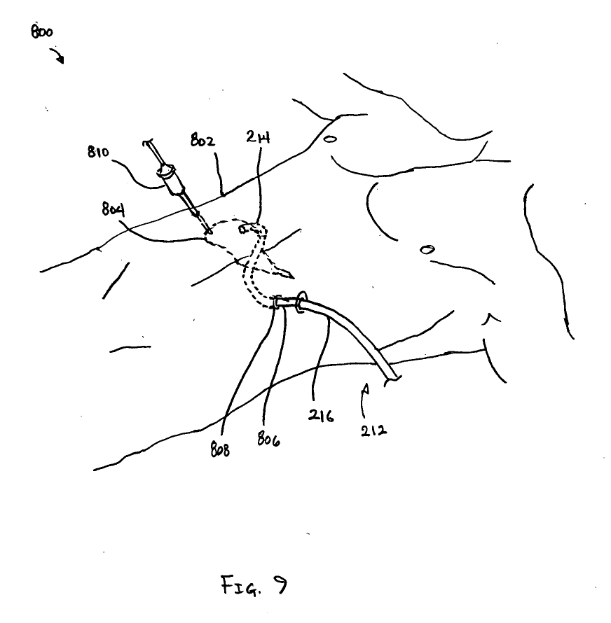

The endoscope device may also be utilized for procedures within the peritoneal cavity. Potential applications may include minimally invasive surgery for urologic, bariatric, and liver surgery. Moreover, minimally invasive access may be achieved for treatments in spinal or orthopedic surgery as well. FIG. 9 shows an example of a procedure 800 within the peritoneal cavity using the endoscopic device 212. The endoscope 212 may be introduced into patient 802 through an incision 808 via a port, which may also function as a datum 806, as described above. The distal portion 214 may be steered to avoid various organs while being advanced to a tissue region to be treated, in this example, the posterior region of liver 804. The distal portion 214 of the endoscope 212 may accordingly be steered while the proximal portion 216 may be automatically controlled to follow a path defined by the distal portion 214 which minimizes contact with the surrounding and adjacent tissue and organs. One or more laparoscopes 810 may optionally be used in combination with the endoscope 212 to assist with the surgical procedure. Once the distal portion 214 is posteriorly positioned of the liver 804, various tools or treatment devices may be advanced through the endoscope 212 from the proximal end to effect the desired treatment. Although this example shows treatment of the liver 804 using the endoscope 212, this is intended to be illustrative and other organs or procedures may be effected using the endoscope 212.

The applications of the devices and methods discussed above are not limited to regions of the body but may include any number of further treatment applications. Other treatment sites may include other areas or regions of the body. Additionally, the present invention may be used in other environments such as exploratory procedures on piping systems, ducts, etc. Modification of the above-described assemblies and methods for carrying out the invention, and variations of aspects of the invention that are obvious to those of skill in the art are intended to be within the scope of the claims.

1. A method of treating an obstructed region of tissue within a body, comprising:
advancing an elongate device into the body through an opening, the elongate device having a proximal portion and a selectively steerable distal portion, the elongate device having a plurality of segments;
selectively steering the distal portion to assume a selected curve along a desired path within the body which avoids contact with tissue; and
further advancing the elongate device through the body and towards the region of tissue to be treated while controlling the proximal portion of the device to assume the selected curve of the distal portion.

2. The method of claim 1 further comprising creating the opening into the body via an incision prior to advancing the elongate device.

3. The method of claim 1 wherein advancing the elongate device comprises percutaneously advancing the elongate device into the body.

4. The method of claim 1 wherein advancing the elongate device comprises advancing the elongate device through the opening defined in a cranium.

5. The method of claim 1 wherein advancing the elongate device comprises advancing the elongate device through the opening defined in a thoracic cavity.

6. The method of claim 1 wherein advancing the elongate device comprises advancing the elongate device through the opening defined in a heart.

7. The method of claim 1 wherein advancing the elongate device comprises advancing the elongate device through the opening defined in an intercostal space.

8. The method of claim 1 wherein advancing the elongate device comprises advancing the elongate device through the opening defined in a peritoneal cavity.

9. The method of claim 1 wherein selectively steering comprises manually steering the distal portion to assume the selected curve.

10. The method of claim 1 wherein selectively steering comprises steering the distal portion through a tortuous path.

11. The method of claim 1 wherein controlling the proximal portion comprises automatically controlling the proximal portion.

12. The method of claim 11 wherein automatically controlling comprises controlling the proximal portion via a computer.

13. The method of claim 1 further comprising advancing the elongate device proximally while controlling the proximal portion of the instrument to assume the selected curve of the distal portion.

14. The method of claim 1 further comprising measuring an axial position change of the elongate device via a datum while advancing the elongate device.

15. The method of claim 1 further comprising measuring a rotational or radial position change of the elongate device via a datum while manipulating the elongate device.

16. The method of claim 1 wherein selectively steering comprises selecting the curve which reduces contact with the tissue.

17. The method of claim 1 wherein selectively steering comprises avoiding contact with organ bodies.

18. The method of claim 1 wherein selectively steering comprises avoiding contact with anatomical structures within the body.

19. The method of claim 1 wherein further advancing the elongate device comprises advancing the device through tissue adjacent to the region of tissue to be treated.

20. The method of claim 1 further comprising treating the region of tissue to be treated.

21. The method of claim 19 wherein treating the region of tissue comprises delivering an instrument to the region of tissue through the elongate device.

22. The method of claim 19 wherein treating the region of tissue comprises treating the region via an apparatus integral with the elongate device.

23. The method of claim 1 further comprising withdrawing the elongate device from the region of tissue.

24. A method of treating a region of tissue within a cranial cavity of a body, comprising:
advancing an elongate body into the cranial cavity, the elongate body having a proximal portion and a selectively steerable distal portion, the elongate body having a plurality of segments;
selectively steering the distal portion to assume a selected curve along a desired path within the body which avoids contact with tissue; and
further advancing the elongate body through the cranial cavity and towards the region of tissue to be treated while controlling the proximal portion of the instrument to assume the selected curve of the distal portion.

25. A method of treating a region of tissue within a thoracic cavity of a body, comprising:
advancing an elongate body into the thoracic cavity, the elongate body having a proximal portion and a selectively steerable distal portion, the elongate body having a plurality of segments;
selectively steering the distal portion to assume a selected curve along a desired path within the body which avoids contact with tissue; and
further advancing the elongate body through the thoracic cavity and towards the region of tissue to be treated while controlling the proximal portion of the instrument to assume the selected curve of the distal portion.

26. A method of treating a region of tissue within a peritoneal cavity of a body, comprising:
advancing an elongate body into the peritoneal cavity, the elongate body having a proximal portion and a selectively steerable distal portion , the elongate body having a plurality of segments;
selectively steering the distal portion to assume a selected curve along a desired path within the body which avoids contact with tissue; and
further advancing the elongate body through the peritoneal cavity and towards the region of tissue to be treated while controlling the proximal portion of the instrument to assume the selected curve of the distal portion.
